(19) United States
(12) Patent Application Publication
(10) Pub. No.: US 2005/0179468 A1
(43) Pub. Date: Aug. 18, 2005

(54) Implementation of MOS capacitor in CT scanner data acquisition system

(76) Inventors: Binling Zhou, Tucson, AZ (US); James L. Todsen, Tucson, AZ (US)

Correspondence Address: TEXAS INSTRUMENTS INCORPORATED, P O BOX 655474, M/S 3999, DALLAS, TX, 75265, US

(21) Appl. No.: 10/779,903

(22) Filed: Feb. 17, 2004

(51) Int. Cl. H03K 5/00; H03K 17/00; G11C 27/02; H03K 19/094; H03K 19/20

(52) U.S. Cl. 326/115

(57) ABSTRACT

An integrator circuit includes an input conductor for conducting an input current, a first amplifier stage having a first input coupled to the input conductor and a second input coupled to receive a reference voltage and a second amplifier stage having a output and an input coupled to an output of the first amplifier stage. An integrating capacitor is coupled between the first input of the first amplifier stage and the output of the second amplifier stage, and a compensation capacitor comprised of an MOS capacitor is coupled between the input and the output of the second amplifier stage. The integrator circuit is especially adapted for use in a CT scanner data acquisition system.

### BACKGROUND OF THE INVENTION

The present invention relates generally to circuitry for CT (computed tomography) scanner data acquisition systems, and more particularly, the present invention relates to circuitry improvements that make it possible to provide a large number of input channels on a single integrated circuit that includes multiple front-end integrators each coupled to receive an input current produced by a corresponding photosensor such as a photodiode receiving light produced by a scintillator in response to x-rays.

A typical CT scanner data acquisition system includes, for each photodiode of a photodiode array, a corresponding front-end integrator that converts the output current of the photodiode to a corresponding output voltage and holds that voltage during sampling thereof by means of a sample/hold circuit. The CT scanner data acquisition system also includes high-resolution ADCs (analog-to-digital converters). A scintillator is typically placed in front of the photodiode array to convert x-rays to light which the photodiodes then converts to a corresponding current.

FIG. 1 shows the configuration of a typical CT scanner system 1 including a large number N (e.g., several thousand) photodiodes upon which light 4 produced by a scintillator in response to X-rays impinge, and also includes a large number of data acquisition systems 2-1 through 2-N/2. Each CT scanner data acquisition system 2 includes four front-end integrators and an ADC. CT scanner system 1 also includes a computer and display system 5. More economical implementation of CT scanner system 1 requires being able to provide more front-end integrator channels and ADCs on a single chip than has been achievable.

Typically, four front-end integrators have been provided on a single integrated circuit chip. The assignee's Burr-Brown DDC112 dual 20-bit current input analog-to-digital converter product is used in data acquisition systems of conventional CT scanners. The DDC112 product is substantially described in commonly owned U.S. Pat. No. 5,841,310, entitled “Current-to-Voltage Integrator for Analog-to-Digital Converter and Method” by Kalthoff et al., issued Nov. 24, 1998 and incorporated herein by reference.

FIG. 2 shows a block diagram of a conventional two-channel data acquisition system 6 sampled by one delta-sigma ADC15 including two identical front-end integrators 2A-1 and 2A-2 used for integrating the input current Iin produced by photodiode D-1, and also including two identical front-end integrators 2B-1 and 2B-2 for photodiode D-2, respectively, in response to light 4 (FIG. 1) produced in response to X-rays impinging on the scintillator. The input of front-end integrators 2A-1 and 2A-2 is connected by a conductor 10-1 to the anode of photodiode D-1, and the input of front-end integrators 2B-1 and 2B-2 is connected by a conductor 10-2 to the anode of photodiode D-2. The common output 14 of the four front-end integrators 2A-1, 2A-2, 2B-1 and 2B-2 is connected to the input of delta sigma ADC 15. As explained in detail in above-mentioned U.S. Pat. No. 5,841,310, the two front-end integrators 2A-1 and 2A-2 or 2B-1 and 2B-2 are operated to integrate the photodiode current signals during alternating first and second time frames in order to provide continuous integration of photodiode output current signal Iin.

Front-end integrator 2A-1 includes a conventional non-inverting amplifier 11 having its (−) input connected to receive photodiode output current signal Iin via conductor 10-1 and its (+) input connected to a reference conductor as shown. The output of non-inverting amplifier 11 is connected by a conductor 12 to the input of a class A inverting amplifier 13, the output of which is connected to output conductor 14. A large capacitance polycrystalline silicon (“poly”) compensation capacitor Cc is connected between conductors 12 and 14 to provide frequency compensation for front-end integrator 2A-1. A much smaller poly integration capacitor Cint is connected between conductor 10-1 and output conductor 14. The switches ensure that only one integrator is connected to input current Iin and the delta-sigma ADC at a time. The structure of front-end integrator 2A-2 is the same as front-end integrator 2A-1. The bandwidth of front-end integrator 2A-1 is limited by the capacitance of compensation capacitor Cc, one plate of which is formed by means of a first polycrystalline silicon layer on an integrated circuit chip and the other plate of which is formed by means of a second polycrystalline silicon layer on the integrated circuit chip.

So-called “poly” capacitors are widely utilized in the manufacture of integrated circuits because their capacitance vs. voltage characteristics (C-V characteristics) are very constant and linear. Unfortunately, the physical size of compensation capacitor Cc is a substantial or major portion of the entire size of the entire front-end integrator 2A-1 including operational amplifier 11, inverting amplifier 13 , compensation capacitor Cc, and integrating capacitor Cint. The variation in capacitance of a conventional poly capacitor with respect to the voltage across the capacitor typically is very low, approximately only 5-15 ppm/V (parts per million per V). Although the low variation in the capacitance of a poly capacitor is very desirable, the very large physical size of a large value poly compensation capacitor Cc greatly limits the number of the front-end integrators that can be provided on a practical integrated circuit chip of economic size, and therefore greatly increases the cost of making a high-resolution CT scanner system having, for example, thousands of photodiode detectors. Furthermore, the amount of chip area required to implement the prior art CT scanner data acquisition circuitry cannot be significantly reduced without unacceptably increasing the amount of noise generated by the prior art data acquisition circuitry, because the bandwidth of the noise is inversely proportional to the capacitance of compensation capacitor Cc. This has made it impractical to provide a large number of front-end integrators in a single integrated circuit chip.

For future-generation CT scan or systems in which the number of detector photodiodes will be greatly increased, e.g., to tens of thousands of photodiodes, however, the manufacturers of CT scanners have a strong need to avoid increasing the sizes of printed circuit boards on which the CT scanner data acquisition systems are provided.

Thus, there is a need for improved CT scanner data acquisition circuitry that greatly reduces the number of integrated circuit chips required to manufacture a high resolution CT scanner system.

There also is an unmet need for an improved front-end integrator circuit for a data acquisition system that requires much less integrated circuit chip area than the prior art.

There also is an unmet need for an improved front-end integrator circuit for a CT scanner data acquisition system that requires much less integrated circuit chip area than the prior art.

There also is a need for an improved CT scanner data acquisition circuit configuration that avoids the need for a trade-off between of the amount of chip area required and the amount of noise generated by the circuit.

### SUMMARY OF THE INVENTION

Accordingly, it is an object of the invention to provide an improved CT scanner data acquisition circuit that greatly reduces the number of integrated circuit chips required to manufacture a-fast, economically feasible high resolution CT scanner system.

It is another object of the invention to provide an improved data acquisition circuit configuration that avoids the need for a trade-off between of the amount of chip area required and the amount of noise generated by the circuit.

It is another object of the invention to provide an improved CT scanner data acquisition circuit configuration that avoids the need to trade off the amount of chip area required against the amount of noise generated by the circuit in order to make a more economically feasible CT scanner.

It is another object of the invention to provide an improved front-end integrator circuit for a data acquisition system that requires much less integrated circuit chip area than the prior art.

It is another object of the invention to provide an improved front-end integrator circuit for a CT scanner data acquisition system that requires much less integrated circuit chip area than the prior art.

Briefly described, and in accordance with one embodiment, the present invention provides an integrator circuit (30) including an input conductor for conducting an input current (Iin), an amplifier stage having an input coupled to the input conductor, an integrating capacitor (Cint) coupled between the input of the amplifier stage and an output of the amplifier stage, and an MOS capacitor (20) coupled between an output (14) of the amplifier stage (13) and a voltage conductor for biasing the MOS capacitor.

In the described embodiment, the integrator circuit (30) includes a first amplifier stage (11) having an input coupled to the input conductor, a second amplifier stage (13) having an output (14) and also having an input coupled to an output (12) of the first amplifier stage (11), an integrating capacitor (Cint) coupled between the input of the first amplifier stage (11) and the output (14) of the second amplifier stage (13), and an MOS compensation capacitor (20) coupled between the input (12) and output (14) of the second amplifier stage (13). The first amplifier stage (11) includes an input stage (11 A) having an output coupled to an input of a folded cascode stage (11B), an output (12) of the folded cascode stage (11B) being coupled to a first terminal of the MOS capacitor (20), a second terminal of the MOS capacitor (20) being coupled to the output (14) of the second amplifier stage (13). The first (11) and second (13) amplifier stages co-act to establish bias voltage across the MOS capacitor (20) so as to bias the MOS capacitor (20) in its accumulation region (C-ACC) for low values of the input current (Iin) to provide a high value of compensation capacitance for the integrator circuit (30) and so as to bias the MOS capacitor (20) in its inversion region (C-INV) for high values of the input current (Iin) to provide a low value of compensation capacitance for the integrator circuit (30). The input current (Iin) is a photodiode current containing a relatively low amount of inherent shot noise for the low values of the input current (Iin) and containing a higher amount of shot noise for the high values of the input current (Iin), and wherein an amount of noise produced by the integrator circuit (30) when the value of the compensation capacitance is high is masked by the higher amount of photodiode noise. The MOS compensation capacitor (20) includes an N-channel source region (23-S) and an N-channel drain region (23-D) both coupled to the input of the second stage amplifier, and also includes a gate (24) disposed over a channel region (23-C) between the N-channel source region and the N-channel drain region, the gate being coupled to the output (14) of the second amplifier stage (13). The second amplifier stage is an inverting class A amplifier. A plurality of the integrator circuits are included in a CT scanner data acquisition system wherein the input current (Iin) is a photodiode current containing a relatively low amount of noise for the low values of the input current (Iin) and containing a higher amount of noise for the high values of the input current (Iin), wherein an amount of noise produced by the integrator circuit (30) when the value of the compensation capacitance is high is masked by the higher amount of noise.

The method of the invention includes operating an integrator circuit conducting an input current into an input of an amplifier stage, charging an integrating capacitor (Cint) coupled between the input and an output of the amplifier stage in response to the input current, and compensating the integrator circuit by controlling the bandwidth of the integrator circuit by biasing an MOS capacitor (20) coupled to the output (14) into a predetermined operating region range.

### BRIEF DESCRIPTION OF THE DRAWINGS

FIG. 1 is a simplified block diagram of a prior art CT scanner data acquisition system.

FIG. 2 is a block diagram of a two-channel data acquisition channel of the system shown in FIG. 1 including four front-end integrators and a delta-sigma ADC.

FIG. 3 is a simplified schematic circuit diagram of a front-end integrator of the present invention.

FIG. 4 is a simplified section view of an MOS capacitor connected as the compensation capacitor Cc of the front-end integrator shown in FIG. 3.

FIG. 5 is a graph illustrating a C-V characteristic of a conventional MOS capacitor.

FIG. 6 is a graph illustrating the amount of photodiode noise and integrator noise produced by the front-end integrator of FIG. 3 as a function of its output voltage Vout.

FIG. 7 is a detail schematic diagram showing the circuitry of the front-end integrator of FIG. 3.

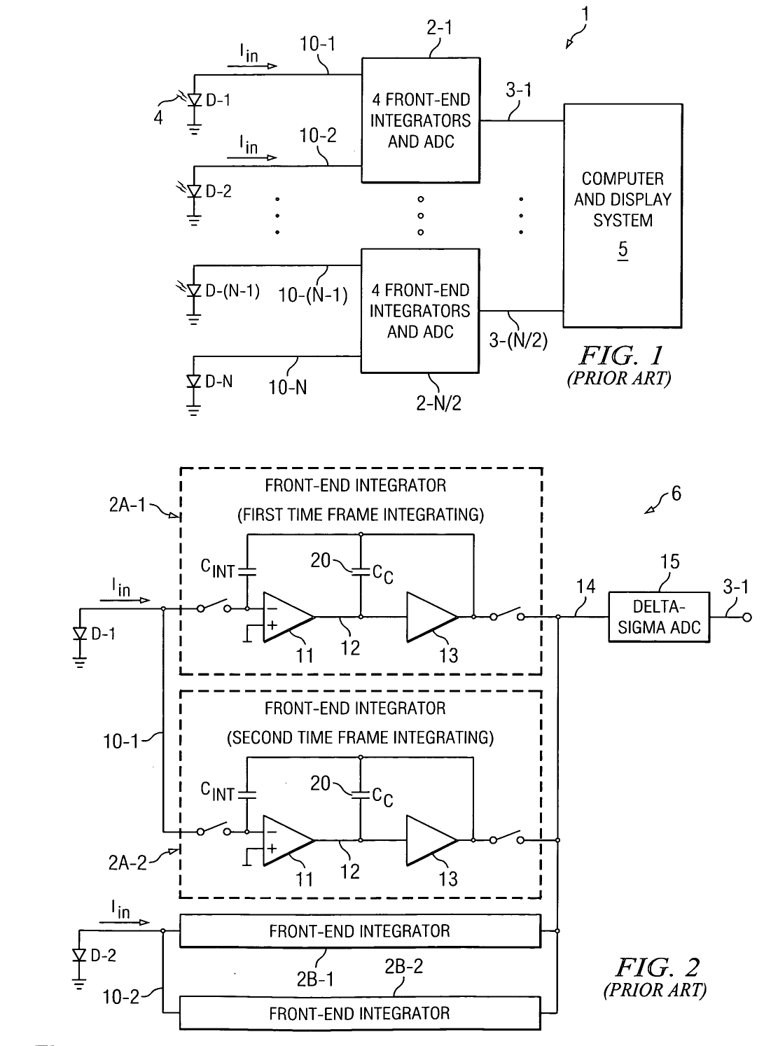

### DETAILED DESCRIPTION OF THE PREFERRED EMBODIMENTS

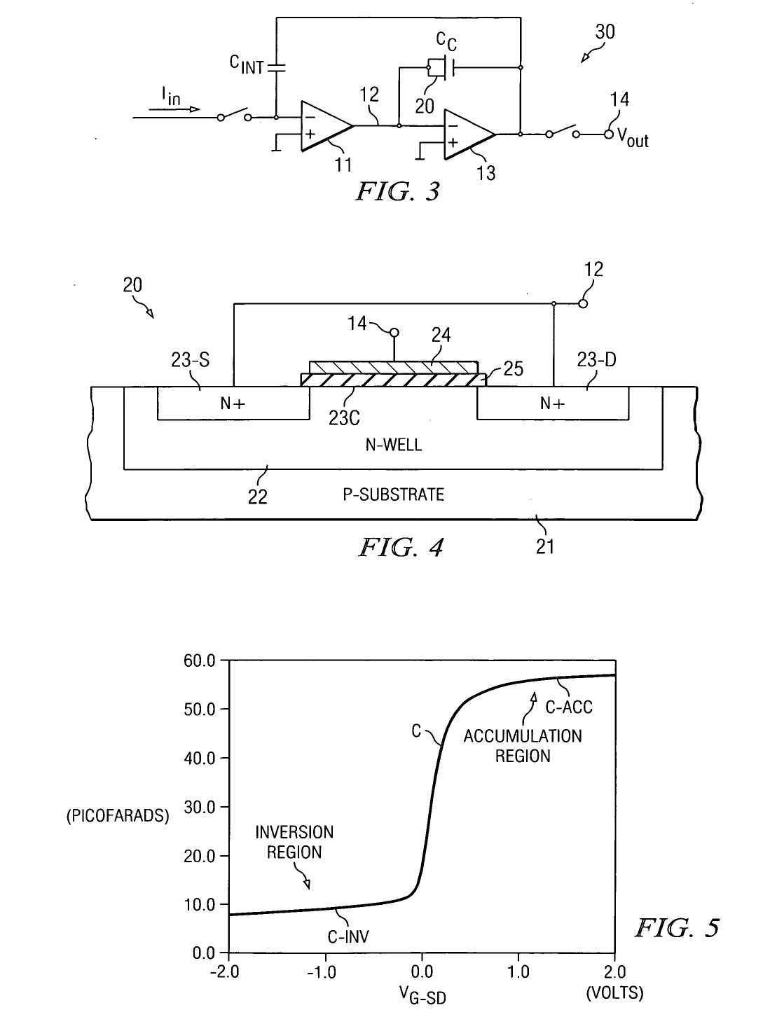

FIG. 3 shows an improved front-end integrator 30 which may be substituted for prior art front-end integrators 2A-1, 2A-2, 2B-1 and 2B-2 in the two-channel data acquisition system of FIG. 2. The only difference between the structure of front-end integrator 30 of FIG. 3 and front-end integrator 2A-1 of FIG. 2 is that in improved front-end integrator 30 the poly compensation capacitor Cc of FIG. 2 has been replaced by an N-channel MOS compensation capacitor 20, also referred to as MOS compensation capacitor Cc, the capacitance of which is also indicated by the symbol Cc. (A P-channel MOS compensation capacitor also might be usable in some cases.) The switches ensure that only one integrator is connected to input current Iin and the delta-sigma ADC at a time.

FIG. 4 shows a section view which shows the structure of N-channel MOS compensation capacitor 20. MOS compensation capacitor 20 as shown in FIG. 4a includes an N-channel “well” region 22 formed in a P-type substrate 21. An N+ source region 23-S and an N+ drain region are formed in N-type well region 22, separated by a “channel” region 23-C. A thin oxide layer 25 is disposed over channel area 23-C, and a doped, conductive polycrystalline silicon gate region 24 is formed on gate oxide 25. The common source region 23-S and drain region 23-D are connected together by conductor 12. (Note that MOS capacitor 20 has a structure quite similar to that of an N-channel transistor having its source and drain regions electrically connected together.) As subsequently explained, MOS capacitor 20 can be connected in accordance with the present invention to function as a variable-capacitance compensation capacitor Cc shown in front-end integrator 30, even though the values of amplifier compensation capacitor is ordinarily should be as in variable as possible.

MOS compensation capacitor 20 in FIG. 4 has a much larger capacitance per unit of chip area than a poly capacitor, because the thickness of the dielectric oxide of a poly capacitor (which, for example, may be approximately 400 angstroms) between the polycrystalline silicon plates of a poly capacitor is much greater the thickness of the dielectric oxide of an MOS capacitor (which, for example, may be only about 130 angstroms) between the gate and body of the MOS capacitor. Unlike the CV characteristic of a poly capacitor, the C-V characteristic curve of an MOS capacitor is very nonlinear, as shown by curve C in FIG. 5. FIG. 4 shows the accumulation region indicated by section C-ACC of curve C which results from majority carriers being induced in channel region 23-C as a result of the gate-to-source/drain voltage V_{G-SD} being greater than approximately 0.5 volts which varies with different manufacturing processes. The “inversion region” of characteristic curve C in FIG. 5 is indicated by section C-INV, and it can be seen that the variable MOS capacitor capacitance Cc is many times greater in the accumulation region C-ACC than in the inversion region C-ThW. Also, the C-V curve of MOS compensation capacitor 20 is shifted slightly compared to the C-V curve of a similarly-configured conventional N-channel MOS transistor.

The nonlinear characteristic wherein the capacitance Cc of MOS capacitor 20 is a very strong function of the voltage V_{G-SD} between its gate electrode and its source and drain electrodes makes it very unsuitable for use as a capacitor in most circuits. For example, the use of an MOS capacitor in most analog circuits would undesirably result in very non-linear circuit operation. For example, if integrating capacitor Cint were to be implemented with an MOS capacitor, the linearity of the amplifier circuit would be very poor.

When MOS capacitor 20 is utilized in accordance with the present invention to implement compensation capacitor Cc, front-end integrator 30 biases MOS compensation capacitor 20 so it is in its accumulation region C-ACC during the low end of the range of Iin values received from the photodiode in order to provide a high capacitance needed to accomplish the required compensation as subsequently explained. For the high end of the range of Iin the values, front-end integrator 30 biases MOS compensation capacitor 20 so that it is in its inversion region C-INV which results in a lower value of compensation capacitance Cc.

FIG. 7 is a schematic diagram of a practical implementation of front-end integrator 30, which is essentially the same as shown in FIG. 3 of above incorporated-by-reference U.S. Pat. No. 5,841,310. In FIG. 7, front-end integrator 30 includes both above-mentioned non-inverting amplifier 11 and class A inverting amplifier 13. Amplifier 11 includes a differential input stage 11A and a folded cascode stage 11B. The output of differential input stage 11A is connected to folded cascode stage 11B, which includes constant current sources 61 and 62 connected to the sources of P-channel cascode transistors 60 and 57, respectively. Their drains are connected to the gate and drain of N-channel transistor 49 and the drain of N-channel transistor 58, respectively. The sources of transistors 49 and 58 are connected to ground. The gates of transistors 49 and 58 are connected together so they form a current mirror. The drain of current mirror output transistor 58 is connected to output conductor 12. The (+) input 28-1 is connected to ground and the (−) input 27-1 is connected to receive the output current produced by the photodiode.

Class A inverting amplifier 13 includes an N-channel transistor 59 having its source connected to ground, its gate connected to output conductor 12, and its drain coupled by output conductor 14 to one terminal of a current source 63, the other terminal of which is connected to +VDD. The differential input stage 11A and its operation are more fully described in commonly assigned U.S. Pat. No. 4,901,031 (Kalthoff et al.).

Front-end integrator 30 also includes a differential auto-zeroing stage 51 which includes auto-zeroing capacitors 31-1 and 31-2 connected between ground and the (+) and (−) auto-zeroing inputs, respectively. The (−) input of auto-zeroing stage 51 is connected to the gate of N-channel source follower transistor 65, and the (+) input is connected to the gate of N-channel source follower transistor 64. The source followers drive the gates of a pair of source-coupled N-channel transistors. Above mentioned switch 33-1 couples output conductor 14 to the inverting input (+) of auto-zeroing stage 51, and switch 34-1 couples V_{REF} to the non-inverting input (−) of auto-zeroing stage 51. (The auto-zeroing technique is well known and therefore is not described herein.) The output conductor 14 of front-end integrator 30 is fed back to an inverting input of auto-zeroing stage 51, which also has its non-inverting input (−) coupled to the reference voltage to stabilize the operational amplifier during the precharging and to cause the output 14 of front-end integrator 30 to be at the reference voltage at the beginning of the integration cycle. Therefore, as can be seen by referring to FIG. 7, the disconnected output 14 of front-end integrator 30 is forced to be equal to the +V_{REF} voltage being applied to the (+) input of auto-zeoring stage 51 during the auto-zeroing operation.

Conductor 25 conducts a bandwidth control signal MA, that controls a switch 54 coupled between one terminal of variable compensation capacitor Cc and conductor 12. The switch 54 is closed during the integration phase to reduce the bandwidth of the integrator and is opened during the sampling phase performed by delta sigma ADC 15 to decrease the settling of the sampling operation. Conductor 12 is connected to the drains of transistors 57 and 58. The other terminal of variable compensation capacitor Cc is connected to output conductor 14. Another capacitor Cm is connected between conductors 12 and 14 to make the integrator stable during the sampling phase performed by delta sigma ADC 15 and may have a capacitance of approximately 35 picofarads, which is much smaller than the maximum value of variable compensation capacitor Cc, which may have a maximum capacitance of approximately 200 picofarads. Note that other types of ADCs, e.g., SAR (successive approximation register) ADCs, could be utilized instead of delta sigma ADCs.

Still referring to FIG. 7, front-end integrator 30 operates such that when the photodiode current signal received by (−) and conductor 27-1 is 0, then the output of the integrator is at VREF, which is typically 4 volts. That voltage is inversely proportional to the photodiode current signal being integrated, so when the photodiode current signal is increased, the output voltage of the integrator at conductor 14 decreases proportionately to the input current. There is a reset feature in the front end integrator, just as described in above mentioned '310 patent.

Input 12 of class A inverting amplifier 13 of front-end integrator circuit 30 typically is maintained at 1 MOS threshold voltage above ground, which is about +1 volt, due to the same gate bias voltage VB2 applied to transistors 57 and 60 and mirror transistors 48 and 49, and the output voltage Vout on conductor 14 typically swings between approximately +1 volt and +4 volts. When the output 12 of class A inverting amplifier 13 is approximately 4 volts, the V_{G-S/D} bias voltage across MOS compensation capacitor Cc is approximately +3 volts, which is well into the accumulation region C-ACC (FIG. 5) of MOS compensation capacitor Cc. But when the photodiode current signal Iin is increased to approximately 50 percent of full-scale, the output voltage 14 of front-end integrator circuit 30 is decreased from approximately 4 volts to approximately +2 volts, so compensation capacitor Cc is biased at approximately +1 volt. With Iin at approximately 50 percent of its full-scale value, there is much less need for a large value of Cc than is the case when Iin is substantially lower.

A key point of this invention is that in CT, the system noise can increase for large signals and not affect overall accuracy. This allows the use of a MOS capacitor for compensation since the DC bias on the MOS capacitor (and hence the MOS capacitor's value) changes in such a way that it decreases for large signals. The light that the photodiode receives contains shot noise, which is proportional to signal magnitude. Therefore, the inherent noise from the photodiode is proportional to signal magnitude. Large signals will have large noise values easing the requirements on the integrator circuits when measuring large signals.

To summarize, an MOS capacitor is used which has a capacitance that is a function of the DC bias across the MOS capacitor. In the described embodiment, the DC bias is a function of the photodiode output current signal level, so the MOS capacitor, which has much higher capacitance per unit of integrated circuit chip area than conventional integrated circuit capacitors, is used as the compensation capacitor wherein its DC bias changes as a function of the photodiode output current signal level. This results in higher bandwidth of the front-end integrator, but the higher front-end integrator bandwidth is then acceptable because the resulting higher noise is overwhelmed and therefore masked by the simultaneous much higher photodiode shot noise.

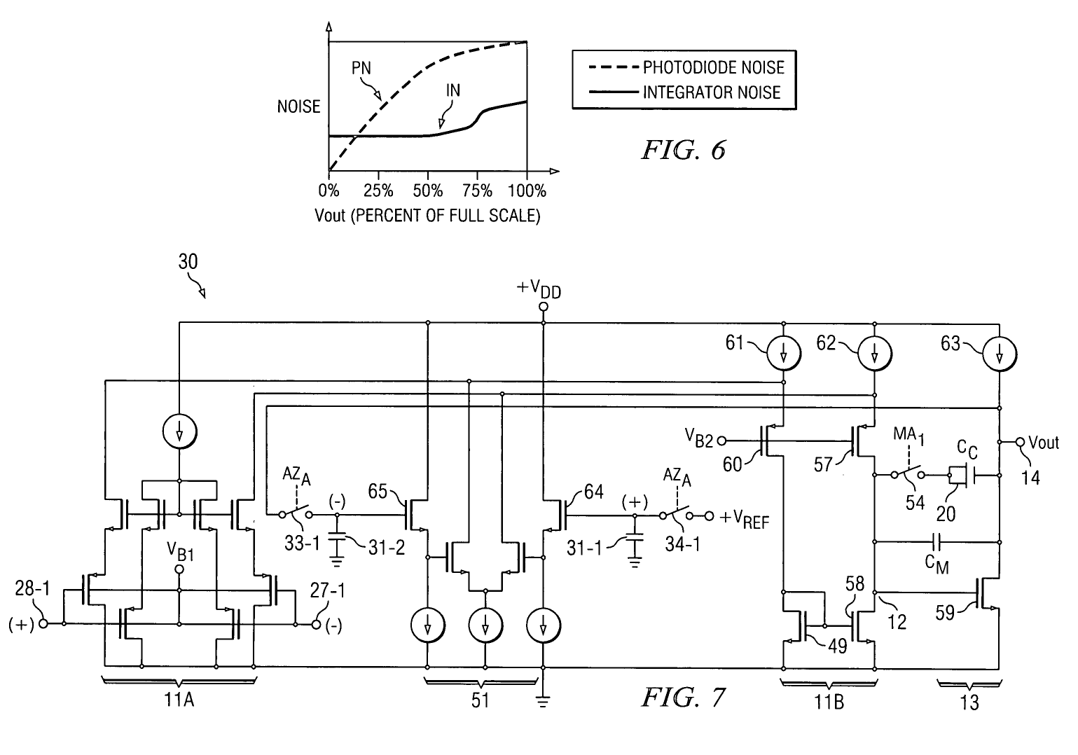

For low values of input current, a typical user of front-end integrator 30 wants low noise operation. But when the input current Iin has increased to approximately one-half of its full-scale value, the user can tolerate the noise produced by front-end integrator 30 because the photodiode “shot” noise is very high. This is in contrast to the general situation, in which it is usually desirable that the noise produced by a compensated amplifier be stable. Note from FIG. 6 than that the photodiode noise increases rapidly as Vout increases above 0 percent of full-scale.

Thus, the variable capacitance of compensation capacitor Cc as shown in FIG. 5 is used to selectively vary the bandwidth of the front-end integrator 30, in contrast to the normal use of a compensation capacitor to stabilize the output of an amplifier, wherein the capacitance of the compensation capacitor should be constant. Due to the configuration of front-end integrator 30, the capacitance Cc of MOS compensation capacitor 20 moves out of its accumulation region (FIG. 5) when the photodiode output current signal Iin is greater than approximately one-half of its full-scale value, causing more noise to be generated by the front-end integrator 30 as its bandwidth is increased. Also, as shown by the curve PN in FIG. 6, the noise produced by the photodiodes increases as the photodiode current increases and thereby causes Vout to increase. However, even with the utilization of the higher-noise MOS compensation capacitor 20, the total amount of noise is dominated by the noise generated by the photodiodes for high values of the photodiode signal current Iin.

In the CT application, the goal is for the noise level of the electronics and integrated circuitry needs to remain below the noise level of the photodiode output current signal in order for the CT system performance not to be degraded by the electronics performance. The noise level of the photodiode output current signal increases with the signal amplitude, so the overall requirement for the electronic noise of the system can increase with increasing signal level because the inherent noise of the photodiode output current signal is increasing at the same time. This relaxes the noise requirement for large level photodiode output current signals into the integrated circuit, which in the present invention simplifies optimizing of the design of the amplifier. Using a variable bandwidth for the amplifier wherein the bandwidth is very low for low photodiode output current signal levels results in low noise from the electronics, and as the signal level increases the bandwidth of the amplifier is increased. There is more noise, but it is acceptable because the inherent noise of the substantially increased photodiode output current signal also is substantially increased. That allows different feedback compensation techniques to be used for low-amplitude and high-amplitude photodiode output current signals.

When the photosensor output current is very low, is necessary to have a very low noise from the electronics so very low front-end integrator bandwidth is required, which necessitates a high value compensation capacitance. The output voltage of front-end integrator 30 is pre-charged to VREF during auto-zeroing so that the amplifier output level is high for a low photodiode output current, and the large bias voltage across the MOS compensation capacitor results in a large value of compensation capacitance Cc which results in low bandwidth, which limits the amount of noise to its minimum value. As the photodiode output current signal magnitude increases, the output voltage of the front-end integrator 30 decreases because of the integrating of the photodiode output current signal, and this decreases, the bias voltage across the MOS compensation capacitor decreases. The compensation capacitance therefore decreases, increasing the amplifier bandwidth, which results in higher noise. However, the photodiode output current signal at that point has a much higher noise content than at lower signal levels, and this masks out the electronic noise, making it irrelevant.

Use of MOS compensation capacitor Cc, which is only approximately one third of the physical size of a poly compensation capacitor, as it is biased and utilized in front-end integrator 30 provides the same amount of effective compensation capacitance as the physically much larger poly compensation capacitor of the prior art at the low photodiode current levels because of the way MOS compensation capacitor 20 is biased to selectively operate in its accumulation region. This reduces the amount of physical chip area required for compensation capacitance by a factor of approximately three.

Thus, the invention provides an improved front-end integrator configuration that requires much less chip area than the prior art, and therefore makes it possible to provide many more data acquisition channels within a single integrated circuit chip.

While the invention has been described with reference to several particular embodiments thereof, those skilled in the art will be able to make various modifications to the described embodiments of the invention without departing from its true spirit and scope. It is intended that all elements or steps which are insubstantially different from those recited in the claims but perform substantially the same functions, respectively, in substantially the same way to achieve the same result as what is claimed are within the scope of the invention. Although each CT scanner data acquisition system 2 is shown including 4 front-end integrators, future generations of the described embodiment of the invention may have 16 or more front-end integrators in each CT scanner data acquisition system. For example, an MOS compensation capacitor could be connected to a single stage amplifier to provide bandwidth control. In one implementation, the gate of an N-channel MOS capacitor can be connected to the output of an amplifier such as a single stage amplifier and the source-drain terminal of the MOS capacitor can be connected to a suitable reference voltage or control voltage.

1. An integrator circuit comprising:
(a) an input conductor for conducting an input current;
(b) an amplifier stage having an input coupled to the input conductor;
(c) an integrating capacitor coupled between the input of the amplifier stage and an output of the amplifier stage; and
(e) an MOS capacitor coupled between an output of the amplifier stage and a voltage conductor for biasing the MOS capacitor.

2. An integrator circuit comprising:
(a) an input conductor for conducting an input current;
(b) a first amplifier stage having an input coupled to the input conductor;
(c) a second amplifier stage having an output and also having an input coupled to an output of the first amplifier stage;
(d) an integrating capacitor coupled between the input of the first amplifier stage and the output of the second amplifier stage; and
(e) an MOS compensation capacitor coupled between the input and output of the second amplifier stage.

3. The integrator circuit of claim 2 wherein the first amplifier stage includes an input stage having an output coupled to an input of a folded cascode stage, an output of the folded cascode stage being coupled to a first terminal of the MOS capacitor, a second terminal of the MOS capacitor being coupled to the output of the second amplifier stage.

4. The integrator circuit of claim 3 wherein the first and second amplifier stages co-act to establish bias voltage across the MOS capacitor so as to bias the MOS capacitor in its accumulation region for low values of the input current to provide a high value of compensation capacitance for the integrator circuit and so as to bias the MOS capacitor in its inversion region for high values of the input current to provide a low value of compensation capacitance for the integrator circuit.

5. The integrator circuit of claim 4 wherein the input current is a photodiode current containing a relatively low amount of noise for the low values of the input current and containing a higher amount of noise for the high values of the input current, and wherein an amount of noise produced by the integrator circuit when the value of the compensation capacitance is high is masked by the relatively high amount of noise.

6. The integrator circuit of claim 2 wherein the first amplifier stage is a non-inverting amplifier stage and the second amplifier stage is an inverting amplifier stage.

7. The integrator circuit of claim 2 wherein the MOS compensation capacitor includes an N-channel source region and an N-channel drain region both coupled to the input of the second stage amplifier, and also includes a gate disposed over a channel region between the N-channel source region and the N-channel drain region, the gate being coupled to the output of the second amplifier stage.

8. The integrator circuit of claim 7 wherein the integrating capacitor is a poly capacitor.

9. The integrator circuit of claim 2 wherein the input of the first amplifier stage conducts a single-ended input signal.

10. The integrator circuit of claim 2 wherein the input of the first amplifier stage conducts a differential input signal.

11. The integrator circuit of claim 6 wherein the second stage amplifier is an inverting class A amplifier.

12. A CT scanner data acquisition system comprising:
(a) a plurality of integrator circuits, each including
i. an input conductor for conducting an input current,
ii. a first amplifier stage having an input coupled to the input conductor,
iii. a second amplifier stage having an output and also having an input coupled to an output of the first amplifier stage,
iv. an integrating capacitor coupled between the input of the first amplifier stage and the output of the second amplifier stage, and
v. an MOS compensation capacitor coupled between the input and output of the second amplifier stage;
(b) a plurality of photodiodes each having an anode coupled to an input conductor of an integrator circuit, respectively;
(c) a plurality of analog-to-digital converters, inputs of the analog-to-digital converters being coupled to the outputs of various integrator circuits.

13. The CT scanner data acquisition system of claim 12 wherein the first amplifier stage includes an input stage having an output coupled to an input of a folded cascode stage, an output of the folded cascode stage being coupled to a first terminal of the MOS capacitor, a second terminal of the MOS capacitor being coupled to the output of the second amplifier stage.

14. The CT scanner data acquisition system of claim 13 wherein the second amplifier stages co-act to establish bias voltage across the MOS capacitor so as to bias the MOS capacitor in its accumulation region for low values of the input current to provide a high value of compensation capacitance for the integrator circuit and so as to bias the MOS capacitor in its inversion region for high values of the input current to provide a low value of compensation capacitance for the integrator circuit.

15. The integrator circuit of claim 14 wherein the input current is a photodiode current containing a relatively low amount of noise for the low values of the input current and containing a relatively high amount of noise for the high values of the input current, wherein an amount of noise produced by the integrator circuit when the value of the compensation capacitance is high is masked by the relatively high amount of noise.

16. The CT scanner data acquisition system of claim 12 wherein the analog-to-digital converters are delta-sigma analog-to-digital converters.

17. The CT scanner data acquisition system of claim 12 wherein the inputs of the analog-to-digital converters are coupled to common outputs of groups of the integrator circuits, respectively.

18. The CT scanner data acquisition system of claim 12 wherein the first amplifier stage is an operational amplifier stage and the second amplifier stage is an inverting amplifier stage.

19. The CT scanner data acquisition system of claim 12 wherein the MOS compensation capacitor includes an N-channel source region and an N-channel drain region both coupled to the input of the second stage amplifier, and also includes a gate disposed over a channel region between the N-channel source region and the N-channel drain region, the gate being coupled to the output of the second amplifier stage.

20. The CT scanner data acquisition system of claim of 19 wherein the integrating capacitor is a poly capacitor.

21. The CT scanner data acquisition system of claim 12 wherein the input of the first amplifier stage conducts a single-ended input signal.

22. The CT scanner data acquisition system of claim 12 wherein the input of the first amplifier stage conducts a differential input signal.

23. The CT scanner data acquisition system of claim 12 wherein the second stage amplifier is an inverting class A amplifier.

24. A method of operating an integrator circuit comprising:
(a) conducting an input current into an input of an amplifier stage;
(b) charging an integrating capacitor coupled between the input and an output of the amplifier stage in response to the input current; and
(c) compensating the integrator circuit by controlling the bandwidth of the integrator circuit by biasing an MOS capacitor coupled to the output into a predetermined operating region range.

25. A method of operating an integrator circuit, comprising:
(a) conducting an input current into an input conductor of a first amplifier stage;
(b) coupling an input of a second amplifier stage to an output of the first amplifier stage;
(c) charging an integrating capacitor coupled between the input of the first amplifier stage and an output of the second amplifier stage; and
(e) compensating the integrator circuit by controlling the bandwidth of the integrator circuit by biasing an MOS compensation capacitor coupled between the input and output of the second amplifier stage into a predetermined operating region range.

26. A method of operating a CT scanner data acquisition system, comprising:
(a) in each of a plurality of integrator circuits,
i. conducting an input current into an input conductor of a first amplifier stage,
ii. coupling an input of a second amplifier stage to an output of the first amplifier stage,
iii. charging an integrating capacitor coupled between the input of the first amplifier stage and an output of the second amplifier stage; and
iv. compensating the integrator circuit by controlling the bandwidth of the integrator circuit by biasing an MOS compensation capacitor coupled between the input and output of the second amplifier stage into a predetermined operating region range;
(b) coupling an anode of each of a plurality of photodiodes to an input conductor of a group of integrator circuits, respectively;
(c) coupling inputs of a plurality of analog-to-digital converters to the outputs of various groups of integrator circuits, respectively.
